(19) United States
(12) Patent Application Publication
(10) Pub. No.: US 2024/0211832 A1
(43) Pub. Date: Jun. 27, 2024

(54) METHODS AND SYSTEMS FOR CREATING AND SCALING INFORMAL CARE NETWORKS

(71) Applicant: KONINKLIJKE PHILIPS N.V., Eindhoven (NL)

(72) Inventors: Nathalie Magali Danielle Dessaud, Eindhoven (NL); Joep van Berkel, Eindhoven (NL); Jan Tatousek, Eindhoven (NL); Chaitanya Dongre, Hamburg (DE)

(21) Appl. No.: 18/544,711

(22) Filed: Dec. 19, 2023

Related U.S. Application Data
(60) Provisional application No. 63/434,664, filed on Dec. 22, 2022.

(51) Int. Cl. G06Q 10/0631; G06Q 10/0639; G16H 10/60; G16H 40/20

(52) U.S. Cl. CPC G06Q 10/063112; G06Q 10/06398; G16H 10/60; G16H 40/20

(57) ABSTRACT

The present disclosure is directed to systems and methods of establishing a patient care network that enables high-quality and safe involvement of selected caregivers in the care of patients receiving virtual or at-home care. As described herein, the methods comprise: identifying a first informal care recipient; identifying an informal caregiver candidate based on caregiver information; assigning the informal caregiver candidate to the first informal care recipient; evaluating a performance of the informal caregiver candidate; determining a certification classification/information for the informal caregiver candidate; and creating and/or storing a caregiver record of the informal caregiver candidate in an informal care database.

### CROSS-REFERENCE TO RELATED APPLICATIONS

This patent application claims the priority benefit under 35 U.S.C. § 119(e) of U.S. Provisional Application No. 63/434,664, filed on Dec. 22, 2022, the contents of which are herein incorporated by reference.

### FIELD OF THE DISCLOSURE

The present disclosure relates generally to methods and systems for creating and/or scaling informal caregiving networks, and more specifically to methods and systems for establishing location-based networks of trained, high-quality, informal caregivers.

### BACKGROUND

Currently in the field of healthcare, there is a major trend of shifting care away from the hospital setting to a home setting. Many factors and challenges in healthcare are contributing to the changing patterns in healthcare needs and demands, such as the growing number of people in need of long-term healthcare, rising costs of care, and relative shortage of healthcare resources and healthcare professionals. This trend of shifting where care is provided is reflected in many aspects of healthcare, including the scaling-up of virtual care, increasing patient self-management, and more intensive focus on building-up and utilizing health services provided by the local community.

As a result, the organization and provision of care outside of a hospital setting faces significant challenges in coordinating affordable care resources, including but not limited to, providing short-term care to patients recently released from a hospital or other clinical setting. For example, such patients may require support in one or more activities outside of the clinical setting for several days or several weeks. In many scenarios, support for these patients can be provided by an individual within the patient's social circle, even where the individual does not have any formal medical training.

While solutions exist where an individual can advertise their availability for at-home assistance and others can engage the services of such individuals, these solutions suffer from many drawbacks. For example, existing solutions are not suitable for integration into clinical patient flows, lack oversight from the primary care provider, and cannot be readily scaled-up for large patient populations, among other drawbacks.

### SUMMARY OF THE DISCLOSURE

According to an embodiment of the present disclosure, a method of establishing a patient care network using an informal care networking system is provided. The method comprises: identifying a first informal care recipient based on a recommendation from a care provider and/or patient information extracted from electronic medical records of the first informal care recipient stored in a patient records database of the informal care networking system; identifying an informal caregiver candidate based on caregiver information extracted from the electronic medical records of the first informal care recipient stored in the patient records database of the informal care networking system; assigning the informal caregiver candidate to the first informal care recipient in connection with one or more informal care tasks using the informal care system based on satisfying one or more assignment criteria; evaluating a performance of the informal caregiver candidate in connection with the one informal care tasks; determining certification information for the informal caregiver candidate based on whether the performance of the informal caregiver candidate in connection with the one or more informal care tasks has met one or more competency criteria associated with the one or more informal care tasks; and storing, in an informal care database of the informal care networking system, a caregiver record of the informal caregiver candidate, wherein the record comprises an identifying information of the informal caregiver candidate, the one or more informal care tasks performed for the first informal care recipient by the informal caregiver candidate, the performance of the informal caregiver candidate in connection with the one or more informal care tasks, and the certification information determined for the informal caregiver candidate.

In an aspect, the method further comprises: receiving, via a care provider interface of the informal care networking system, an input comprising the recommendation from the care provider, wherein the recommendation identifies one or more patients an informal care recipient.

In an aspect, the method further comprises: extracting patient information from the electronic medical records of the first informal care recipient stored in the patient records database of the informal care networking system; and extracting caregiver information from the electronic medical records of the first informal care recipient stored in the patient records database of the informal care networking system.

In an aspect, the patient information identifies one or more informal care tasks that the first informal care recipient will need assistance performing.

In an aspect, the caregiver information identifies one or more untrained individuals associated with the first informal care recipient who are able to assist the first informal care recipient in performing the one or more informal care tasks.

In an aspect, the method further comprises: identifying at least a second informal care recipient based on a recommendation from a care provider and/or patient information extracted from the electronic medical records of at least the second informal care recipient stored in the patient records database of the informal care networking system; identifying an existing informal caregiver candidate based on caregiver information extracted from the caregiver records stored in the informal care database; and assigning, using the informal care networking system, the existing informal caregiver candidate to the second informal care recipient in connection with one or more informal care tasks based on satisfying one or more assignment criteria.

In an aspect, the assignment criteria comprise a travel radius for the existing informal caregiver candidate, and wherein the second informal care recipient satisfies the assignment criteria if the second informal care recipient lives within the travel radius of the existing informal caregiver candidate.

In an aspect, the assignment criteria comprise a competency requirement for the one or more informal care tasks to be performed in connection with the second informal care recipient, and the existing informal caregiver candidate satisfies the assignment criteria if the existing informal caregiver candidate meets the competency requirement for the one or more informal care tasks to be performed in connection with the second informal care recipient.

In an aspect, the method further comprises: determining whether the second informal care recipient satisfies the assignment criteria by determining whether the second informal care recipient lives within the travel radius of the existing informal caregiver candidate based on patient information extracted from electronic medical records of the second informal care recipient stored in the patient records database of the informal care networking system; and determining whether the existing informal caregiver candidate satisfies the assignment criteria by determining whether the existing informal caregiver candidate meets the competency requirement for the one or more informal care tasks to be performed in connection with the second informal care recipient based on caregiver records stored in the informal care database of the informal care networking system.

In an aspect, the method further comprises: transmitting, via an informal caregiver interface of the informal caregiver networking system, an offer for additional training to the existing informal caregiver candidate in connection with one or more additional care tasks, wherein the one or more additional care tasks are different than the one or more informal care tasks; receiving, via the informal caregiver interface of the informal caregiver networking system, an indication that the additional training has been completed by the existing informal caregiver candidate in connection with the one or more additional care tasks; updating, in the informal care database of the informal care networking system, the caregiver record for the existing informal caregiver candidate to include competency information in connection with the one or more additional care tasks.

In an aspect, the method further comprises: identifying at least a third informal care recipient based on a recommendation from a care provider and/or patient information extracted from the electronic medical records of at least the third informal care recipient stored in the patient records database of the informal care networking system; assigning, using the informal care networking system, the existing informal caregiver candidate to the third informal care recipient in connection with the one or more additional care tasks based on satisfying one or more assignment criteria.

According to another embodiment of the present disclosure, an informal care networking system is provided. The system comprises: a patient records database comprising electronic medical records for a plurality of patients; an informal care database comprising informal caregiver information for a plurality of informal caregiver candidates; and one or more processors in communication with the patient records database and the informal care database, wherein the one or more processors are configured to: identify at least a first informal care recipient based on a recommendation from a care provider and/or patient information extracted from electronic medical records of the first informal care recipient stored in the patient records database; identify at least a first informal caregiver candidate based on caregiver information extracted from the electronic medical records of the first informal care recipient stored in the patient records database; assign the first informal caregiver candidate to the first informal care recipient in connection with one or more informal care tasks based on satisfying one or more assignment criteria; evaluate a performance of the first informal caregiver candidate in connection with the one informal care tasks; determine certification information for the first informal caregiver candidate based on whether the performance of the first informal caregiver candidate in connection with the one or more informal care tasks has met one or more competency criteria associated with the one or more informal care tasks; and store, in the informal care database, a caregiver record of the first informal caregiver candidate, wherein the record comprises an identifying information of the first informal caregiver candidate, the one or more informal care tasks performed for the first informal care recipient by the first informal caregiver candidate, the performance of the first informal caregiver candidate in connection with the one or more informal care tasks, and the certification information determined for the first informal caregiver candidate.

In an aspect, the one or more processors are further configured to: identify at least a second informal care recipient based on a recommendation from a care provider and/or patient information extracted from the electronic medical records of at least the second informal care recipient stored in the patient records database; identify an existing informal caregiver candidate based on caregiver information extracted from the caregiver records stored in the informal care database; and assign the existing informal caregiver candidate to the second informal care recipient in connection with one or more informal care tasks based on satisfying one or more assignment criteria.

In an aspect, the system further comprises an informal care recipient interface configured to receive patient-reported outcomes associated with the one or more informal care tasks performed by the informal caregiver candidate.

In an aspect, the system further comprises an informal caregiver interface configured to: (i) present additional training to an existing informal caregiver candidate in connection with one or more additional care tasks; and (ii) track completion of the additional training by the existing informal caregiver candidate in connection with the one or more additional care tasks.

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart illustrating a method of establishing a patient care network according to aspects of the present disclosure.

FIG. 2A is a flowchart illustrating a method of establishing a patient care network according to further aspects of the present disclosure.

FIG. 2B is a flowchart illustrating a method of establishing a patient care network according to still further aspects of the present disclosure.

FIG. 3 is a diagram illustrating an informal care networking system according to aspects of the present disclosure.

FIG. 4 is a diagram illustrating an informal care networking module according to aspects of the present disclosure.

### DETAILED DESCRIPTION OF EMBODIMENTS

It is appreciated by the present disclosure that as more healthcare needs and services are shifted outside formal care settings (e.g., outside of a hospital or other clinical setting), these informal care services need to be positioned as an extension of traditional care models and adapted over time to improve patient outcomes, including but not limited to, costs, clinical outcomes, patient-reported outcomes, and the like. Further, as these healthcare needs and services are shifted outside formal care settings, the need to engage individuals who can meet the needs of these patients in their particular homes and communities becomes a challenge. While attempts to provide on-demand at-home assistance have been made, these solutions are not suitable for integration into clinical patient flows, lack oversight from the primary care provider, and cannot be readily scaled-up for large patient populations, among other drawbacks.

Accordingly, provided herein are systems and methods of establishing a patient care network that enable high-quality and safe involvement of selected caregivers in the care of patients receiving virtual or at-home care, reduce workload of mobile care professionals, reduce costs, improves outcomes, and patient satisfaction. Further, the systems and methods described herein enable seamless transition from a formal care setting where a patient is supported by healthcare professionals to an informal care setting where the patient is supported by one or more informal caregivers. The systems and methods described herein further enable seamless scaling of the informal caregiver network such that informal caregivers can be allocated to patients outside of their immediate patient contacts.

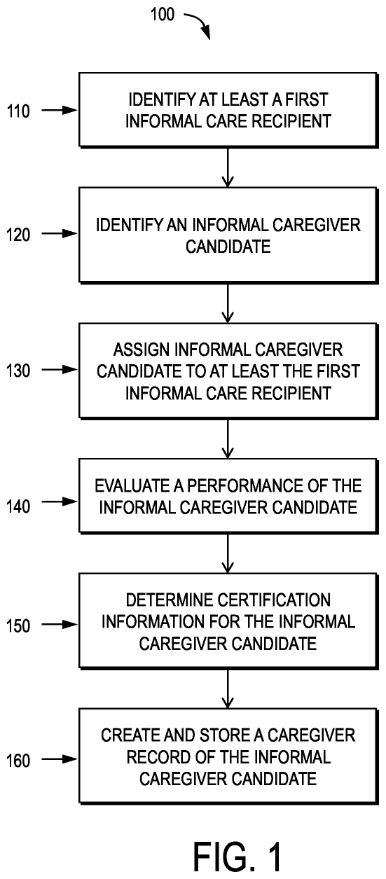

With reference to FIG. 1, a method 100 of establishing a patient care network is provided. In embodiments, the method 100 comprises: in a step 110, identifying a first informal care recipient; in a step 120, identifying an informal caregiver candidate based on caregiver information; in a step 130, assigning the informal caregiver candidate to the first informal care recipient; in a step 140, evaluating a performance of the informal caregiver candidate; in a step 150, determining a certification classification/information for the informal caregiver candidate; and, in a step 160, creating and/or storing a caregiver record of the informal caregiver candidate in an informal care database. These and other aspects are discussed in more detail below.

In the step 110, the method 100 includes identifying a first informal care recipient. As used herein, the term “informal care recipient” refers to a patient from a formal care setting (e.g., a hospital or other clinical setting) who will require informal care services in connection with one or more tasks after leaving the formal care setting. As used herein, the term “informal care” refers to care that is provided by an individual without formal medical training as opposed to an individual with formal medical training (e.g., doctor, nurse, etc.).

In embodiments, the step 110 can include identifying at least a first informal care recipient, such as one or more informal care recipients. In some embodiments, an informal care recipient may be identified at least in part based on a recommendation from a formal care provider, such as a care provider providing healthcare services to the patient in a formal care setting.

In further embodiments, an informal care recipient may be identified at least in part based on patient information extracted from the electronic medical records of the informal care recipient. Patient information can include, but is not limited to, information such as identification information (e.g., date of birth, name, marital status, social security number, etc.), medical history (e.g., allergies, treatments, medical care, past and present diagnoses, habits such as diet, alcohol intake, exercise, etc.), medication information, family history, treatment history (e.g., complaints, history of illness, vital signs, physical exams, surgical history, immunization history), medical directives, lab results (e.g., lab results related to cells, tissues, body fluids, or imaging tests), consent forms, and the like. In embodiments, the electronic medical records of the patient may be stored in a patient records database, such as a hospital medical records database.

In the step 120, the method 100 includes identifying an informal caregiver candidate based on caregiver information. As used herein, the term “informal caregiver” refers to an individual without formal medical training but suitable for providing assistant in one or more care-related tasks to another individual in an informal care setting. As such, the term “informal caregiver candidate” refers to an individual that is a candidate to be an informal caregiver for one or more informal care recipients.

In embodiments, the step 120 can include identifying at least a first informal caregiver candidate, such as one or more informal caregiver candidates. In some embodiments, an informal caregiver candidate can be identified based on caregiver information, as mentioned above. Caregiver information can include, but is not limited to, identification information (e.g., date of birth, name, marital status, social security number, etc.), education information (e.g., degrees earned, graduation year), occupation history, history of providing informal care, and the like.

In embodiments, caregiver information may be collected or otherwise extracted from the electronic medical records of an informal care recipient. For example, in some embodiments, medical records for a patient identified as a potential informal care recipient may be used to identify one or more family members with the potential skills to provide informal care to the patient in an informal care setting. In other embodiments, the caregiver information may be collected or otherwise extracted from caregiver records stored in an informal care database, as discussed in more detail below.

In the step 130, the method 100 includes assigning the informal caregiver candidate to the first informal care recipient. That is, in embodiments, the step 130 includes assigning one or more informal caregiver candidates identified in step 120 to one or more informal care recipients identified in step 110. In some embodiments, assigning an informal caregiver candidate to an informal care recipient can include automatically generating an electronic notification and/or agreement between the caregiver candidate and care recipient that outlines the terms of the care to be provide (e.g., times, days, tasks, conditions, etc.). In embodiments, the assigning an informal caregiver candidate to an informal care recipient can include updating an informal care database storing records of the informal caregiver candidate and/or the informal care recipient to include details of the care provided or to be provided to the care recipient (e.g., times, days, tasks, conditions, etc.).

In embodiments, an informal caregiver candidate may be assigned to an informal care recipient in connection with one or more informal care tasks. That is, an informal caregiver candidate may be selected and assigned to an informal care recipient to support or otherwise assist the informal care recipient with one or more informal care tasks. In embodiments, an informal care task can include, but is not limited, keeping up with personal hygiene (e.g., showering, etc.), performing one or more exercises, taking medications, and/or the like.

In embodiments, an informal caregiver candidate may be assigned to an informal care recipient based on satisfying one or more assignment criteria. The assignment criteria can include one or more caregiver-specific criteria and/or one or more care recipient-specific criteria. In embodiments, the assignment criteria can include a maximum distance (e.g., travel distance, travel time, etc.) between the informal caregiver candidate and the care recipient. For example, the caregiver may have a rule that they can assist care recipients within a 20-mile radius or 30-minute travel time radius. In embodiments, the caregiver candidate may also have specific criteria or preferences for the types of care recipients, the conditions of the location of services (e.g., will there be pets or other animals present, etc.), and/or the like. In embodiments, the care recipient may also have specific criteria or preferences for the types of informal care candidates, the level of experience, and/or the like.

In particular embodiments, the assignment criteria includes at least an indication that the informal caregiver candidate is willing to assist potential care recipients with one or more identified informal care tasks. In further embodiments, the assignment criteria includes at least an indicate that the informal caregiver candidate has the competences required to assist potential care recipients with the one or more identified informal care tasks. In still further embodiments, the assignment criteria includes one or more of: patient clinical interactions or behaviors, health status, social determinants of health (SDoH), general practitioner (GP) assessment, patient self-measurement skills, patient self-sufficiency level and/or the like.

In the step 140, the method 100 includes evaluating a performance of the informal caregiver candidate. In embodiments, the performance of the informal caregiver candidate is evaluated based on the quality of the recipient-caregiver interactions using applicable quality metrics. In particular embodiments, these quality metrics can include clinical outcomes, readmission data, patient-reported outcomes, and/or the like.

In the step 150, the method 100 includes determining a certification classification/information for the informal caregiver candidate. More specifically, the step 150 can include certifying that an informal caregiver candidate has met a given care competence criteria. Accordingly, as used herein, a certification classification and/or certification information can include an indicate of whether an informal caregiver candidate has met such care competence criteria. In embodiments, whether an informal caregiver candidate is qualified to obtain a particular certification classification may be determined based on the performance of the informal caregiver candidate with one or more informal care recipients in connection with one or more informal care tasks. For example, depending on the performance of the informal caregiver with one or more informal care recipients, the informal caregiver may be certified as having a competence in one or more skills associated with the one or more informal care tasks and/or a particular skill level (e.g., novice, experienced, expert, etc.) in one or more informal care tasks.

In embodiments, determining a certification classification and/or certification information for an informal caregiver candidate may also include updating, down-grading, and/or revoking a caregiver's certifications. For example, if a gap in the caregiver's experience is revealed through the performance evaluation, then that may be reflected in the caregiver's certification information.

In the step 160, the method 100 includes creating and/or storing a caregiver record of the informal caregiver candidate in an informal care database. In embodiments, the caregiver record can comprise identification information (e.g., name, date of birth, social security number, etc.) for the caregiver and information related to the caregiver's activities in connection with one or more care recipients. More specifically, the caregiver record can comprise, for example and without limitation, an identifying information of the informal caregiver candidate, the one or more informal care tasks performed for an informal care recipient by the informal caregiver candidate, the performance of the informal caregiver candidate in connection with the one or more informal care tasks, and the certification information determined for the informal caregiver candidate.

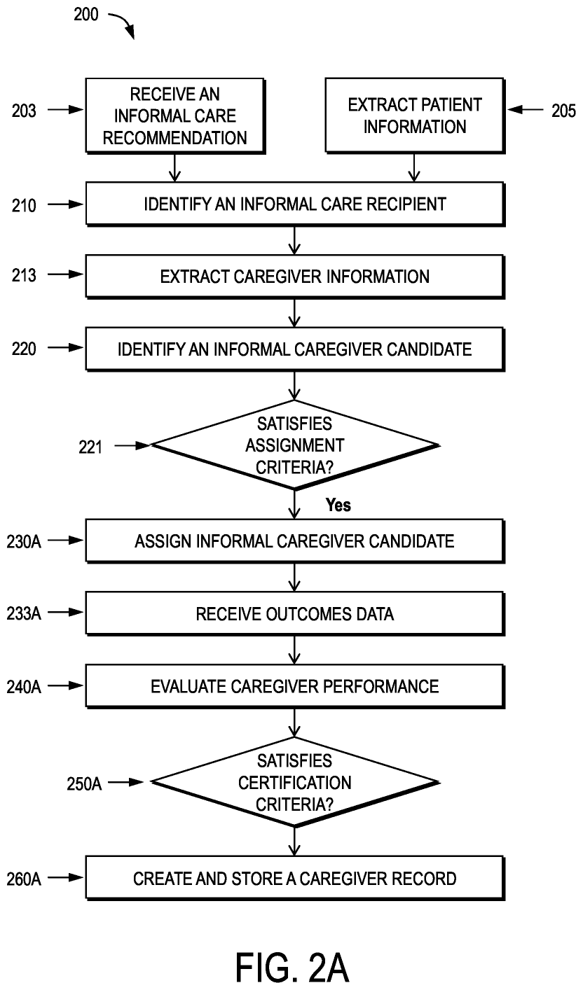

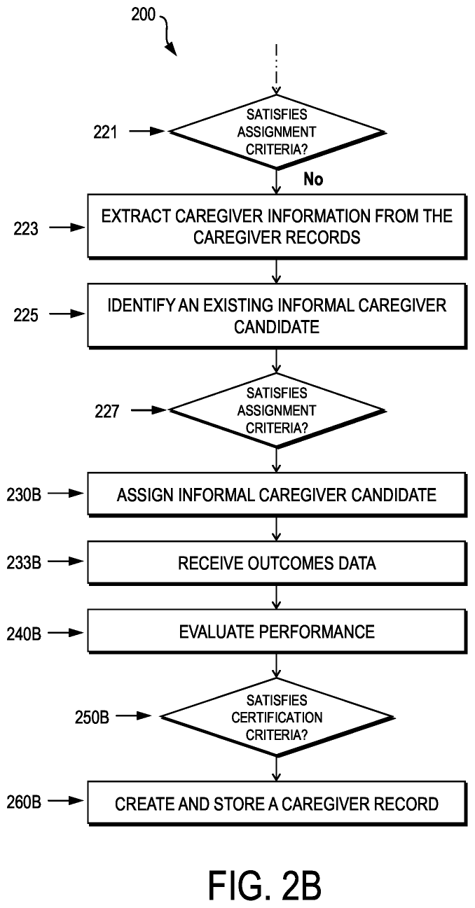

Turning to FIGS. 2A and 2B, a method 200 of establishing a patient care network is provided in accordance with further aspects of the present disclosure. As shown in FIG. 2A, the method 200 comprises: in a step 203, receiving an input comprising an informal care recommendation from a care provider via a care provider interface, wherein the recommendation identifies one or more patients as potential informal care recipients; in a step 205, extracting patient information from the electronic medical records of one or more patients; in a step 210, identifying a first informal care recipient based on either the recommendation received in step 203 and/or the patient information extracted in step 205; in a step 213, extracting caregiver information from the electronic medical records of an informal care recipient; in a step 220, identifying an informal caregiver candidate based on caregiver information extracted in step 213; in a step 221, determining whether an informal caregiver candidate and a potential informal care recipient satisfy one or more assignment criteria; in a step 230A, if the assignment criteria are satisfied, assigning the informal caregiver candidate to the informal care recipient; in a step 233A, receiving outcomes data; in a step 240A, evaluating a performance of the informal caregiver candidate; in a step 250A, determining a certification classification/information for the informal caregiver candidate; and, in a step 260A, creating and/or storing a caregiver record of the informal caregiver candidate in an informal care database. These and other aspects are discussed in more detail below.

In the step 203, the method 200 can include receiving an input comprising an informal care recommendation from a care provider via a care provider interface, wherein the recommendation identifies one or more patients as potential informal care recipients. In embodiments, the recommendation may be received from a general practitioner or other healthcare specialist associated with the one or more patients (e.g., in a clinical setting).

In the step 205, the method 200 can include extracting patient information from the electronic medical records of one or more patients. As described above, the patient information can include, but is not limited to, information such as identification information (e.g., date of birth, name, marital status, social security number, etc.), medical history (e.g., allergies, treatments, medical care, past and present diagnoses, habits such as diet, alcohol intake, exercise, etc.), medication information, family history, treatment history (e.g., complaints, history of illness, vital signs, physical exams, surgical history, immunization history), medical directives, lab results (e.g., lab results related to cells, tissues, body fluids, or imaging tests), consent forms, and the like. In embodiments, the patient information can be extracted from electronic medical records of one or more patients (including a patient identified as a potential informal care recipient) stored in an electronic medical records database using natural language processing and/or a machine learning algorithm.

In embodiments, step 203 and step 205 may be performed simultaneously or sequentially. In some embodiments, step 203 may be performed and step 205 may not be performed. In other embodiments, step 205 may be performed and step 203 may not be performed.

In the step 210, the method 200 can include identifying at least a first informal care recipient, such as one or more informal care recipients. In some embodiments, an informal care recipient may be identified at least in part based on a recommendation from a formal care provider received in step 203. In further embodiments, an informal care recipient may be identified at least in part based on patient information extracted from the electronic medical records of the informal care recipient in step 205.

In the step 213, the method 200 can include extracting caregiver information from the electronic medical records of a patient identified as a potential informal care recipient. As described above, caregiver information can include, but is not limited to, identification information (e.g., date of birth, name, marital status, social security number, etc.), education information (e.g., degrees earned, graduation year), occupation history, history of providing informal care, and the like. In embodiments, the caregiver information can be extracted from electronic medical records of one or more patients (e.g., a patient identified as a potential informal care recipient) stored in an electronic medical records database using natural language processing and/or a machine learning algorithm. For example, in some embodiments, medical records for a patient identified as a potential informal care recipient may be used to identify one or more family members with the potential skills to provide informal care to the patient in an informal care setting.

In the step 220, the method 200 can include identifying an informal caregiver candidate based on caregiver information extracted in step 213. Then, in the step 221, the method 200 can include determining whether the identified informal caregiver candidate and a potential informal care recipient satisfy one or more assignment criteria. The assignment criteria can include one or more caregiver-specific criteria and/or one or more care recipient-specific criteria. In embodiments, the assignment criteria can include a maximum distance (e.g., travel distance, travel time, etc.) between the informal caregiver candidate and the care recipient. For example, the caregiver may have a rule that they can assist care recipients within a 20-mile radius or 30-minute travel time radius. In embodiments, the caregiver candidate may also have specific criteria or preferences for the types of care recipients, the conditions of the location of services (e.g., will there be pets or other animals present, etc.), and/or the like. In embodiments, the care recipient may also have specific criteria or preferences for the types of informal care candidates, the level of experience, and/or the like.

In particular embodiments, the assignment criteria includes at least an indication that the informal caregiver candidate is willing to assist potential care recipients with one or more identified informal care tasks. In further embodiments, the assignment criteria includes at least an indicate that the informal caregiver candidate has the competences required to assist potential care recipients with the one or more identified informal care tasks. In still further embodiments, the assignment criteria includes one or more of: patient clinical interactions or behaviors, health status, social determinants of health (SDoH), general practitioner (GP) assessment, patient self-measurement skills, patient self-sufficiency level and/or the like.

In the step 230A, if the assignment criteria are satisfied, the method 200 can include assigning the informal caregiver candidate to the informal care recipient. In some embodiments, assigning an informal caregiver candidate to an informal care recipient can include automatically generating an electronic notification and/or agreement between the caregiver candidate and care recipient that outlines the terms of the care to be provide (e.g., times, days, tasks, conditions, etc.). In embodiments, the assigning an informal caregiver candidate to an informal care recipient can include updating an informal care database storing records of the informal caregiver candidate and/or the informal care recipient to include details of the care provided or to be provided to the care recipient (e.g., times, days, tasks, conditions, etc.).

In embodiments, an informal caregiver candidate may be assigned to an informal care recipient in connection with one or more informal care tasks. That is, an informal caregiver candidate may be selected and assigned to an informal care recipient to support or otherwise assist the informal care recipient with one or more informal care tasks. In embodiments, an informal care task can include, but is not limited, keeping up with personal hygiene (e.g., showering, etc.), performing one or more exercises, taking medications, and/or the like.

In the step 233A, the method 200 can include receiving outcomes data from one or more sources. In embodiments, for example, the outcomes data can include information received from an informal care recipient, an informal caregiver candidate, the care provider, a patient interface, a care provider interface, and/or the like. In some embodiments, the outcomes data can include information related to the quality of the recipient-caregiver interactions using applicable quality metrics, such as clinical outcomes, readmission data, patient-reported outcomes, and/or the like.

In the step 240A, the method 200 can include evaluating a performance of the informal caregiver candidate. For example, the performance of an informal caregiver candidate may be evaluated based on the outcomes information received in step 233A.

In the step 250A, the method 200 can include determining a certification classification/information for the informal caregiver candidate. As described above in connection with step 150 of the method 100, the step 250A of the method 200 can include certifying that an informal caregiver candidate has met a given care competence criteria. In embodiments, whether an informal caregiver candidate is qualified to obtain a particular certification classification may be determined based on the performance of the informal caregiver candidate with one or more informal care recipients in connection with one or more informal care tasks. For example, depending on the performance of the informal caregiver with one or more informal care recipients, the informal caregiver may be certified as having a competence in one or more skills associated with the one or more informal care tasks and/or a particular skill level (e.g., novice, experienced, expert, etc.) in one or more informal care tasks.

In the step 260A, the method 200 can include creating and/or storing a caregiver record of the informal caregiver candidate in an informal care database. As described above in connection with step 160, the caregiver record can comprise identification information (e.g., name, date of birth, social security number, etc.) for the caregiver and information related to the caregiver's activities in connection with one or more care recipients. In specific embodiments, the caregiver record can comprise, for example and without limitation, an identifying information of the informal caregiver candidate, the one or more informal care tasks performed for an informal care recipient by the informal caregiver candidate, the performance of the informal caregiver candidate in connection with the one or more informal care tasks, and the certification information determined for the informal caregiver candidate.

With reference to FIG. 2B, a flowchart illustrating additional aspects of a method of establishing a patient care network (e.g., method 200) is shown. In particular, the method 200 can additionally and/or alternatively include: in a step 221, determining that an informal caregiver candidate does not satisfy the assignment criteria for a potential informal care recipient; in a step 223, extracting caregiver information from a plurality of caregiver records stored in an informal care database; in a step 225, identifying an existing informal caregiver based on the extracted caregiver information; in a step 227, determining whether the existing informal caregiver and the potential informal care recipient satisfy one or more assignment criteria; in a step 230B, if the assignment criteria are satisfied, assigning the existing informal caregiver candidate to the informal care recipient; in a step 233B, receiving outcomes data; in a step 240B, evaluating a performance of the existing informal caregiver candidate; in a step 250B, determining a certification classification/information for the existing informal caregiver candidate; and, in a step 260B, updating and/or storing a caregiver record of the existing informal caregiver candidate in the informal care database. These and other aspects are discussed in more detail below.

More specifically, as illustrated in FIG. 2B, it should be appreciated that an informal caregiver candidate may not meet all or some of the assignment criteria in connection with one or more informal care recipients. As such, it is contemplated that a network of existing informal caregivers is created and stored in an informal care database, which may be relied upon to identify additional informal caregiver candidates that may satisfy all or some of the assignment criteria in connection with the one or more informal care recipients.

In particular, in the step 221, it may be determined that the informal caregiver candidate and/or the informal care recipient do not mutually satisfy one or more necessary assignment criteria. For example, the informal caregiver candidate and the informal care recipient may live too far away from one another, or the informal caregiver candidate may not have competency in assisting with the informal care recipient with the one or more informal care tasks needed.

Accordingly, in the step 223, the method 200 can include extracting caregiver information from one or more caregiver records stored in an informal care database. As described herein, the informal care database can comprise a plurality of caregiver records storing caregiver information for a plurality of existing informal caregiver candidates. In particular, these existing informal caregiver candidates may have been identified, certified, and documented in a previous iteration of the methods (e.g., method 100, 200) described herein. Put another way, the plurality of caregiver records analyzed in the step 223 can include caregiver records created and/or stored in steps 160, 260A, 260B, for example.

In embodiments, the caregiver information can be extracted from caregiver records of one or more informal caregiver candidates stored in an informal care database using natural language processing and/or a machine learning algorithm. In some embodiments, the caregiver information can include, but is not limited to, identification information (e.g., date of birth, name, marital status, social security number, etc.), education information (e.g., degrees earned, graduation year), occupation history, history of providing informal care, and the like.

In a step 225, the method 200 can then include identifying an existing informal caregiver based on the extracted caregiver information. Then, in a step 227, the method 200 can include determining whether the existing informal caregiver candidates identified in the step 225 and/or the potential informal care recipient satisfy one or more assignment criteria. As described above, the assignment criteria can include one or more caregiver-specific criteria and/or one or more care recipient-specific criteria, such as a maximum distance (e.g., travel distance, travel time, etc.) between the informal caregiver candidate and the care recipient.

In a step 230B, if the assignment criteria are satisfied, the method 200 can include assigning an existing caregiver candidate to an informal care recipient. In embodiments, assigning an existing informal caregiver candidate to an informal care recipient can include automatically generating an electronic notification and/or agreement between the existing caregiver candidate and care recipient that outlines the terms of the care to be provide (e.g., times, days, tasks, conditions, etc.). In embodiments, assigning an informal caregiver candidate to an informal care recipient can also include updating an informal care database storing records of the existing informal caregiver candidate and/or the informal care recipient to include details of the care provided or to be provided to the care recipient (e.g., times, days, tasks, conditions, etc.).

As described above, an existing informal caregiver candidate may be assigned to an informal care recipient in connection with one or more informal care tasks. That is, an existing informal caregiver candidate may be selected and assigned to an informal care recipient to support or otherwise assist the informal care recipient with one or more informal care tasks. In embodiments, an informal care task can include, but is not limited, keeping up with personal hygiene (e.g., showering, etc.), performing one or more exercises, taking medications, and/or the like.

In a step 233B, the method 200 can include receiving outcomes data from one or more sources, as described with respect to step 233A of the method 200. In a step 240B, the method 200 can include evaluating a performance of the existing informal caregiver candidate, as described with respect to step 240A of the method 200.

Then, in a step 250B, the method 200 can include determining a certification classification/information for the existing informal caregiver candidate. In embodiments, determining a certification classification and/or certification information for an existing informal caregiver candidate may include updating, down-grading, and/or revoking a caregiver's certifications. For example, if a gap in an existing caregiver's experience is revealed through the performance evaluation, then that may be reflected in the existing caregiver's certification information.

In a step 260B, the method 200 can include updating and/or storing a caregiver record of the existing informal caregiver candidate in the informal care database. In embodiments, the step 260B includes modifying an existing caregiver record corresponding to the existing informal caregiver candidate with information related to the existing caregiver's activities in connection with one or more new care recipients. As described above, the caregiver record can comprise, for example and without limitation, an identifying information of the existing informal caregiver candidate, the one or more informal care tasks performed for an informal care recipient by the existing informal caregiver candidate, the performance of the existing informal caregiver candidate in connection with the one or more informal care tasks, and the certification information determined for the existing informal caregiver candidate.

Although the methods 100, 200 have been described herein in connection with certain aspects, it should be appreciated that one or more steps of the methods 100, 200 may be repeated one or more times in order to effectively and efficiently scale-up the informal care network. In some embodiments, for example, one or more steps of the methods 100, 200 may be repeated one or more times for at least a second informal care recipient, including three or more informal care recipients. In further embodiments, one or more steps of the methods 100, 200 may be repeated one or more times for at least a second informal caregiver candidate, including three or more informal caregiver candidates.

Additionally, in some embodiments, the methods 100, 200 described herein can include utilizing one or more user interfaces to offer and/or provide supplemental training, thereby expanding the skillsets of one or more informal caregiver candidates. For example, in some embodiments, the methods 100, 200 can include: transmitting, via an informal caregiver interface of the informal caregiver networking system, an offer for additional training to the existing informal caregiver candidate in connection with one or more additional care tasks, wherein the one or more additional care tasks are different than other one or more informal care tasks (e.g., one or more informal care tasks that the informal caregiver candidate is already competent in); receiving, via the informal caregiver interface of the informal caregiver networking system, an indication that the additional training has been completed by the existing informal caregiver candidate in connection with the one or more additional care tasks; and updating, in the informal care database of the informal care networking system, the caregiver record for the existing informal caregiver candidate to include competency information in connection with the one or more additional care tasks.

Also provided herein are informal care networking systems configured to establish an informal care network. In particular, an informal care networking system of the present disclosure comprises: a patient records database comprising electronic medical records for a plurality of patients; an informal care database comprising informal caregiver information for a plurality of informal caregiver candidates; and one or more processors in communication with the patient records database and the informal care database, wherein the one or more processors are configured to perform one or more steps of the method (e.g., methods 100, 200) described herein.

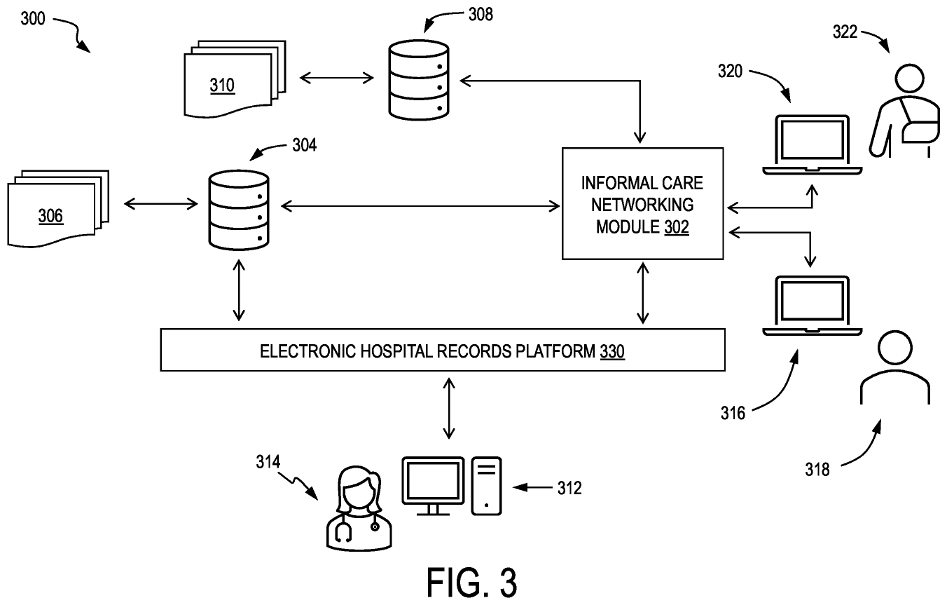

For example, with reference to FIG. 3, an informal care networking system 300 comprising an informal care networking module 302 is illustrated in accordance with certain aspects of the present disclosure. The informal care networking system 300 can be configured to configured to establish an informal care network, as described herein. In some embodiments, the informal care networking module 302 can be at least part of a larger patient data management system (PDMS) and/or another type of patient management solution.

As shown, the informal care networking system 300 can comprise: a patient records database 304 comprising electronic medical records 306 for a plurality of patients; an informal care database 308 comprising informal caregiver information 310 for a plurality of informal caregiver candidates; and one or more processors in communication with the patient records database 304 and the informal care database 308, wherein the one or more processors are configured to perform one or more steps of the methods (e.g., methods 100, 200) described herein.

In embodiments, informal care networking system 300 can also comprise a plurality of formal care provider interfaces 312 associated with one or more formal care providers 314, a plurality of informal caregiver interfaces 316 associated with one or more informal caregivers 318, and a plurality of care recipient interfaces 320 associated with one or more informal care recipients 322. In particular embodiments, an electronic patient data management system 324 may be in communication with the informal care networking module 302 and/or one or more other components 304, 308, 312, 316, 320 of the informal care networking system 300.

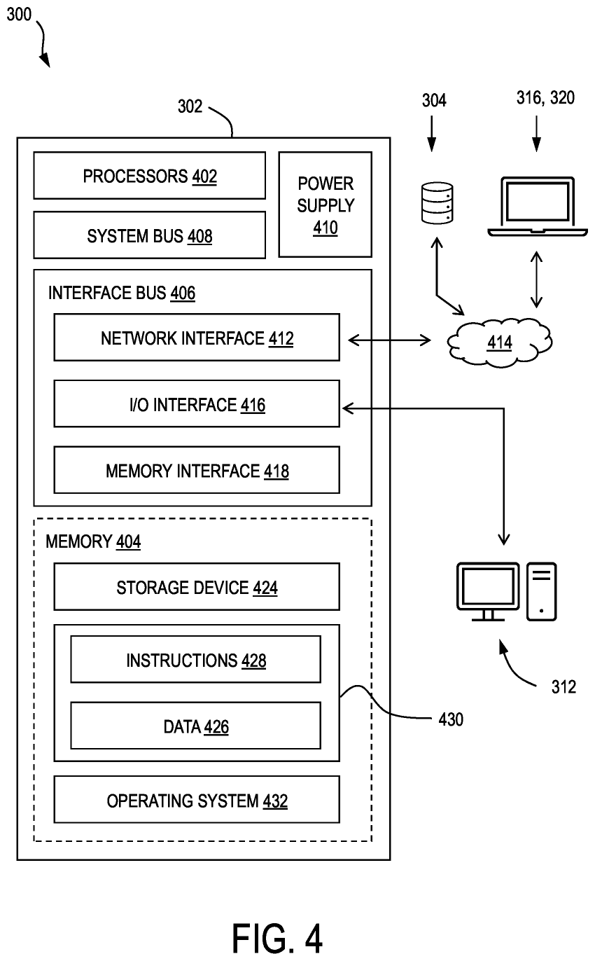

With reference to FIG. 4, the informal care networking module 302 is illustrated in accordance with further aspects of the present disclosure. As shown, informal care networking module 302 may comprise one or more processors 402, machine-readable memory 404, and an interface bus 406, all of which may be interconnected and/or communicate through a system bus 408 containing conductive circuit pathways through which instructions (e.g., machine-readable signals) may travel to effectuate communication, tasks, storage, and the like. The informal care networking module 302 may be connected to a power source 410, which can include an internal power supply and/or an external power supply.

As discussed above, the one or more processors 402 may be configured to perform one or more steps of the methods described herein, including but not limited to, the following: identify at least a first informal care recipient based on a recommendation from a care provider 314 and/or patient information extracted from electronic medical records 306 of the first informal care recipient 322 stored in the patient records database 304; identify at least a first informal caregiver candidate 318 based on caregiver information extracted from the electronic medical records of the first informal care recipient 322 stored in the patient records database 304; assign the first informal caregiver candidate 318 to the first informal care recipient 322 in connection with one or more informal care tasks based on satisfying one or more assignment criteria; evaluate a performance of the first informal caregiver candidate 318 in connection with the one informal care tasks; determine certification information for the first informal caregiver candidate 318 based on whether the performance of the first informal caregiver candidate 318 in connection with the one or more informal care tasks has met one or more competency criteria associated with the one or more informal care tasks; and store, in the informal care database 308, a caregiver record 310 of the first informal caregiver candidate 318, wherein the record 310 comprises an identifying information of the first informal caregiver candidate 318, the one or more informal care tasks performed for the first informal care recipient 322 by the first informal caregiver candidate 318, the performance of the first informal caregiver candidate 318 in connection with the one or more informal care tasks, and the certification information determined for the first informal caregiver candidate 318.

In some examples, the one or more processors 402 may include a high-speed data processor adequate to execute the program components described herein and/or various specialized processing units as may be known in the art. In some examples, the one or more processors 402 may be a single processor, multiple processors, or multiple processor cores on a single die.

In some examples, the interface bus 406 may include a network interface 412 configured to connect the informal care networking module 302 to a communications network 414, an input/output (“I/O”) interface 416 configured to connect and communicate with one or more interfaces 312, and/or a memory interface 418 configured to accept, communication, and/or connect to a number of machine-readable memory devices (e.g., memory 404).

The I/O interface 416 may operatively connect the informal care networking module 302 with one or more devices, including but not limited to external, peripheral, and/or swappable devices.

The network interface 412 may operatively connect the informal care networking module 302 to a communications network 414, which can include a direct interconnection, the Internet, a local area network (“LAN”), a metropolitan area network (“MAN”), a wide area network (“WAN”), a wired or Ethernet connection, a wireless connection, and similar types of communications networks, including combinations thereof. In some examples, the informal care networking module 302 may communicate with one or more remote/cloud-based servers 420, and/or wireless devices via the communications network 414 and the network interface 412.

The memory 404 can be variously embodied in one or more forms of machine-accessible and machine-readable memory. In some examples, the memory 404 includes a storage device 424 comprises one or more types of memory. For example, the storage device 424 can include, but is not limited to, a non-transitory storage medium, a magnetic disk storage, an optical disk storage, an array of storage devices, a solid-state memory device, and the like, including combinations thereof.

Generally, the memory 404 is configured to store data/information 426 and instructions 428 that, when executed by the one or more processors 402, causes the informal care networking module 302 to perform one or more tasks. In particular examples, the memory 404 includes an informal care networking package 530 that comprises a collection of program components, database components, and/or data. Depending on the particular implementation, the patient monitoring transition package 430 may include software components, hardware components, and/or some combination of both hardware and software components.

The informal care networking package 430 may include, but is not limited to, instructions 428 having one or more software packages configured to perform one or more of the steps of the methods (e.g., method 100, 200) described herein. These software packages may be incorporated into, loaded from, loaded onto, or otherwise operatively available to and from the informal care networking module 302.

In some examples, the informal care networking package 430 and/or one or more individual software packages may be stored in a local storage device 424. In other examples, the informal care networking package 430 and/or one or more individual software packages may be loaded onto and/or updated from a remote server 420 via the communications network 414.

In particular embodiments, the informal care networking package 430 can include, but is not limited to, instructions 428 having one or more components configured to perform or facilitate the performance of the methods (e.g., method 100, 200) described herein. These components may be incorporated into, loaded from, loaded onto, or otherwise operatively available to and from the informal care networking module 302.

In embodiments, the informal care networking module 302 may also include an operating system component 432, which may be stored in the memory 404. The operating system component 432 may be an executable program facilitating the operation of the informal care networking module 302. Typically, the operating system component 432 can facilitate access of the I/O interface, network interface, and memory interface, and can communicate with other components of the informal care networking system 300.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein, reference numbers followed by a letter (“A”, “B”, “C”, etc.) are utilized to assist in identification of elements or features similar to those bearing the same base reference number across different embodiments, but while facilitating further discussion with respect to the particular features in separate embodiments. It should be appreciated that features or elements having a base reference numeral appended with a letter are generally arranged and function as described with respect to that element or feature that share the base reference number, except as otherwise indicated.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method of establishing a patient care network using an informal care networking system, the method comprising:
identifying a first informal care recipient based on a recommendation from a care provider and/or patient information extracted from electronic medical records of the first informal care recipient stored in a patient records database of the informal care networking system;
identifying an informal caregiver candidate based on caregiver information extracted from the electronic medical records of the first informal care recipient stored in the patient records database of the informal care networking system;
assigning the informal caregiver candidate to the first informal care recipient in connection with one or more informal care tasks using the informal care system based on satisfying one or more assignment criteria;
evaluating a performance of the informal caregiver candidate in connection with the one informal care tasks;
determining certification information for the informal caregiver candidate based on whether the performance of the informal caregiver candidate in connection with the one or more informal care tasks has met one or more competency criteria associated with the one or more informal care tasks; and
storing, in an informal care database of the informal care networking system, a caregiver record of the informal caregiver candidate, wherein the record comprises an identifying information of the informal caregiver candidate, the one or more informal care tasks performed for the first informal care recipient by the informal caregiver candidate, the performance of the informal caregiver candidate in connection with the one or more informal care tasks, and the certification information determined for the informal caregiver candidate.

2. The method of claim 1, further comprising:
receiving, via a care provider interface of the informal care networking system, an input comprising the recommendation from the care provider, wherein the recommendation identifies one or more patients an informal care recipient.

3. The method of claim 1, further comprising:
extracting patient information from the electronic medical records of the first informal care recipient stored in the patient records database of the informal care networking system; and
extracting caregiver information from the electronic medical records of the first informal care recipient stored in the patient records database of the informal care networking system.

4. The method of claim 3, wherein the patient information identifies one or more informal care tasks that the first informal care recipient will need assistance performing.

5. The method of claim 3, wherein the caregiver information identifies one or more untrained individuals associated with the first informal care recipient who are able to assist the first informal care recipient in performing the one or more informal care tasks.

6. The method of claim 1, further comprising:
identifying at least a second informal care recipient based on a recommendation from a care provider and/or patient information extracted from the electronic medical records of at least the second informal care recipient stored in the patient records database of the informal care networking system;
identifying an existing informal caregiver candidate based on caregiver information extracted from the caregiver records stored in the informal care database; and
assigning, using the informal care networking system, the existing informal caregiver candidate to the second informal care recipient in connection with one or more informal care tasks based on satisfying one or more assignment criteria.

7. The method of claim 6, wherein the assignment criteria comprise a travel radius for the existing informal caregiver candidate, and wherein the second informal care recipient satisfies the assignment criteria if the second informal care recipient lives within the travel radius of the existing informal caregiver candidate.

8. The method of claim 7, wherein the assignment criteria comprise a competency requirement for the one or more informal care tasks to be performed in connection with the second informal care recipient, and the existing informal caregiver candidate satisfies the assignment criteria if the existing informal caregiver candidate meets the competency requirement for the one or more informal care tasks to be performed in connection with the second informal care recipient.

9. The method of claim 8, further comprising:
determining whether the second informal care recipient satisfies the assignment criteria by determining whether the second informal care recipient lives within the travel radius of the existing informal caregiver candidate based on patient information extracted from electronic medical records of the second informal care recipient stored in the patient records database of the informal care networking system; and
determining whether the existing informal caregiver candidate satisfies the assignment criteria by determining whether the existing informal caregiver candidate meets the competency requirement for the one or more informal care tasks to be performed in connection with the second informal care recipient based on caregiver records stored in the informal care database of the informal care networking system.

10. The method of claim 6, further comprising:
transmitting, via an informal caregiver interface of the informal caregiver networking system, an offer for additional training to the existing informal caregiver candidate in connection with one or more additional care tasks, wherein the one or more additional care tasks are different than the one or more informal care tasks;
receiving, via the informal caregiver interface of the informal caregiver networking system, an indication that the additional training has been completed by the existing informal caregiver candidate in connection with the one or more additional care tasks; and
updating, in the informal care database of the informal care networking system, the caregiver record for the existing informal caregiver candidate to include competency information in connection with the one or more additional care tasks.

11. The method of claim 10, further comprising:
identifying at least a third informal care recipient based on a recommendation from a care provider and/or patient information extracted from the electronic medical records of at least the third informal care recipient stored in the patient records database of the informal care networking system;
assigning, using the informal care networking system, the existing informal caregiver candidate to the third informal care recipient in connection with the one or more additional care tasks based on satisfying one or more assignment criteria.

12. An informal care networking system, comprising:
a patient records database comprising electronic medical records for a plurality of patients;
an informal care database comprising informal caregiver information for a plurality of informal caregiver candidates; and
one or more processors in communication with the patient records database and the informal care database, wherein the one or more processors are configured to:
identify at least a first informal care recipient based on a recommendation from a care provider and/or patient information extracted from electronic medical records of the first informal care recipient stored in the patient records database;
identify at least a first informal caregiver candidate based on caregiver information extracted from the electronic medical records of the first informal care recipient stored in the patient records database;
assign the first informal caregiver candidate to the first informal care recipient in connection with one or more informal care tasks based on satisfying one or more assignment criteria;
evaluate a performance of the first informal caregiver candidate in connection with the one informal care tasks;
determine certification information for the first informal caregiver candidate based on whether the performance of the first informal caregiver candidate in connection with the one or more informal care tasks has met one or more competency criteria associated with the one or more informal care tasks; and
store, in the informal care database, a caregiver record of the first informal caregiver candidate, wherein the record comprises an identifying information of the first informal caregiver candidate, the one or more informal care tasks performed for the first informal care recipient by the first informal caregiver candidate, the performance of the first informal caregiver candidate in connection with the one or more informal care tasks, and the certification information determined for the first informal caregiver candidate.

13. The informal care networking system of claim 12, wherein the one or more processors are further configured to:
identify at least a second informal care recipient based on a recommendation from a care provider and/or patient information extracted from the electronic medical records of at least the second informal care recipient stored in the patient records database;
identify an existing informal caregiver candidate based on caregiver information extracted from the caregiver records stored in the informal care database; and
assign the existing informal caregiver candidate to the second informal care recipient in connection with one or more informal care tasks based on satisfying one or more assignment criteria.

14. The informal care networking system of claim 12, further comprising an informal care recipient interface configured to receive patient-reported outcomes associated with the one or more informal care tasks performed by the informal caregiver candidate.

15. The informal care networking system of claim 12, further comprising an informal caregiver interface configured to: (i) present additional training to an existing informal caregiver candidate in connection with one or more additional care tasks; and (ii) track completion of the additional training by the existing informal caregiver candidate in connection with the one or more additional care tasks.
